(19) United States
(12) Patent Application Publication
(10) Pub. No.: US 2023/0213493 A1
(43) Pub. Date: Jul. 6, 2023

(54) METHOD OF CALCULATING FRESHNESS SCORE OF FOOD INGREDIENT

(71) Applicant: TmaxEnterprise Co., Ltd., Gyeonggi-do (KR)

(72) Inventors: Myongsik Gong, Gyeonggi-do (KR); Eunsung Kim, Gyeonggi-do (KR); Youngjin Kim, Gyeonggi-do (KR); Leere Song, Gyeonggi-do (KR)

(21) Appl. No.: 17/679,341

(22) Filed: Feb. 24, 2022

(30) Foreign Application Priority Data
Jan. 6, 2022 (KR) 10-2022-0001899
Feb. 24, 2022 (KR) 10-2022-0024235

(51) Int. Cl. G01N 33/02

(52) U.S. Cl. CPC G01N 33/02

(57) ABSTRACT

An exemplary embodiment of the present disclosure discloses a method of calculating a freshness score of a food ingredient, the method being performed by a server including at least one processor. The method may include: receiving food ingredient information about food ingredients loaded into a moving object from a user terminal; receiving environment information about an environment inside the moving object from the user terminal, the environment information including at least one of temperature data obtained by measuring a temperature inside the moving object and humidity data obtained by measuring humidity inside the moving object; calculating a freshness score of the food ingredient based on the food ingredient information and the environment information; and generating monitoring information to be provided to the user terminal by using the calculated freshness score.

### CROSS-REFERENCE TO RELATED APPLICATIONS

This application claims priority to and the benefit of Korean Patent Application No. 10-2022-0001899 filed in the Korean Intellectual Property Office on Jan. 6, 2022, and Korean Patent Application No. 10-2022-0024235 filed in the Korean Intellectual Property Office on Feb. 24, 2022 the entire contents of which are incorporated herein by reference.

### TECHNICAL FIELD

The present disclosure relates to a method of calculating a freshness score of a food ingredient, and particularly, to a method of calculating a freshness score of a food ingredient by using data measured through a sensor provided inside a moving object.

This work was supported by the Industrial Technology Innovation Program (20003722, Development of Block Chain Platform for Distribution History Management and Product Certification Services) funded by the Ministry of Trade, Industry & Energy (MOTIE, Korea) in 2019.

### BACKGROUND ART

In general, distribution of food ingredients may be made as a process in which a producer produces food and distributes the food to a mart or market, and the like through a distributor, and then a consumer purchases the necessary food. In the distribution of such food ingredients, freshness may be the most important factor for consumers to decide on food ingredients. In the case of food ingredients such as fruits, meat, or fresh foods, freshness may be particularly important, and a serious accident can occur if stale food ingredients reach the consumer's table. Accordingly, sellers who sell food ingredients to marts or markets must always check the distributed food ingredients, and may discard food ingredients that are not fresh.

These food ingredients may have different storage conditions for maintaining freshness for each food ingredient. In particular, temperature and humidity during storage of food ingredients may have a great effect on freshness. However, even if temperature and humidity have a large effect, the existing method for inspecting the freshness of food ingredients can calculate only the score or grade according to the range by simply measuring the temperature or humidity.

### PRIOR ART LITERATURE

[Patent Document]
Korean Patent No. 10-1717594

### SUMMARY OF THE INVENTION

The present disclosure has been conceived in response to the foregoing background art, and has been made in an effort to provide a method of calculating a freshness score for each food ingredient in real time based on the amount of change according to measurement values of a temperature and humidity and time.

The technical objects of the present disclosure are not limited to the foregoing technical objects, and other non-mentioned technical objects will be clearly understood by those skilled in the art from the description below.

In order to implement the foregoing object, an exemplary embodiment of the present disclosure discloses a method of calculating a freshness score of a food ingredient, the method being performed by a server including at least one processor, the method including: receiving food ingredient information about food ingredients loaded into a moving object from a user terminal; receiving environment information about an environment inside the moving object from the user terminal, the environment information including at least one of temperature data obtained by measuring a temperature inside the moving object and humidity data obtained by measuring humidity inside the moving object; calculating a freshness score of the food ingredient based on the food ingredient information and the environment information; and generating monitoring information to be provided to the user terminal by using the calculated freshness score.

The environment information may be received at a predetermined time interval or is received when a predetermined condition is satisfied.

The calculating of the freshness score of the food ingredient based on the food ingredient information and the environment information may include: determining a pre-stored appropriate temperature range and appropriate humidity range corresponding to the food ingredient based on the food ingredient information; and calculating the freshness score of the food ingredient based on the appropriate temperature range, the appropriate humidity range, and the environment information.

The freshness score may be determined by using a rule-based freshness model which calculates a first score based on a ratio in which at least one temperature data is included in the appropriate temperature range and a ratio in which at least one humidity data is included in the appropriate humidity range.

The freshness score may be calculated through a computation of the first score and a first weight preset for the first score.

The rule-based freshness model may calculate the first score based on Equation,

$$i th Inclusion Rate ( \% ) = \frac{\# ( {T}_{lower} \leq {T}_{i} < {T}_{upper} and {H}_{lower} \leq {H}_{i} < {H}_{upper} )}{i} ,$$

and i may be a natural number for indicating the time point or the number of times of the measurement, inclusion rate may be a percentage value for indicating a ratio in which at least one temperature data is included in the appropriate temperature range and a ratio in which at least one humidity data is included in the appropriate humidity range, T_{lower} may be a lower limit value of the appropriate temperature range, Tᵤₚₚₑᵣ may be an upper limit value of the appropriate temperature range, and Tᵢ may be a temperature of the i^{th} measurement, H_{lower} may be a lower limit value of the appropriate humidity range, Hᵤₚₚₑᵣ may be an upper limit value of the appropriate humidity range, and Hᵢ may be humidity of the i^{th} measurement.

The rule-based freshness model may determine a first section corresponding to the i^{th} inclusion rate among the plurality of sections divided into a predetermined number in order to calculate the first score, and calculate the first score based on a rule pre-applied to the first section.

The freshness score may be determined based on a clustering-based freshness model which calculate a second score based on a density in which at least one temperature data is included in the appropriate temperature range and a density in which at least one humidity data is included in the appropriate humidity range.

The freshness score may be calculated through a computation of the second score and a second weight preset for the second score.

The clustering-based freshness model may determine a density-based cluster based on at least one temperature data and at least one humidity data, and calculate the second score based on a ratio of normal data present in the cluster among at least one temperature data and at least one humidity data included in the environment information.

The freshness score may be calculated by using an ensemble model, and the ensemble model may include: a rule-based freshness model which calculates a first score based on a ratio in which at least one temperature data is included in the appropriate temperature range and a ratio in which at least one humidity data is included in the appropriate humidity range; and a clustering-based freshness model which calculates a second score based on a density in which at least one temperature data is included in the appropriate temperature range and a density in which at least one humidity data is included in the appropriate humidity range.

The freshness score may be calculated through a computation of the first score, a first weight preset for the first score, the second score, and a second weight preset for the second score.

The method may further include: receiving feedback information about the freshness score from the user terminal after providing the monitoring information to the user terminal; determining whether to update the first weight and the second weight based on the feedback information; and re-calculating the freshness score when it is determined to update the first weight and the second weight.

The re-calculated freshness score may include: a first freshness score calculated through a computation of the first score, a first-1 weight in which the first weight is updated, the second score, a second-1 weight in which the second weight is updated; and a second freshness score calculated through a computation of the first score, a first-2 weight in which the first weight is updated differently from the first-1 weight, the second score, and a second-2 weight in which the second weight is updated differently from the second-1 weight, and the method may further include generating monitoring information to be provided to the user terminal by using the first freshness score and the second freshness score when the first freshness score and the second freshness score are calculated.

In order to implement the foregoing object, another exemplary embodiment of the present disclosure discloses a method of calculating a freshness score of a food ingredient, the method being performed by a computing device including at least one processor, the method including: receiving food ingredient information about food ingredients loaded into a moving object from a user terminal; receiving environment information about an environment inside the moving object from the user terminal, the environment information including at least one of temperature data obtained by measuring a temperature inside the moving object and humidity data obtained by measuring humidity inside the moving object; calculating a freshness score of the food ingredient based on the food ingredient information and the environment information; and generating monitoring information to be provided to the user terminal by using the calculated freshness score.

The technical solutions obtainable from the present disclosure are not limited to the foregoing solutions, and other non-mentioned solution means will be clearly understood by those skilled in the art from the description below.

According to the exemplary embodiments of the present disclosure, it is possible to provide the method of calculating a freshness score of a food ingredient, which is capable of maintaining a temperature and humidity range appropriate for each food ingredient, and monitoring the temperature and the humidity so that the temperature and the humidity are not sharply changed.

The effects of the present disclosure are not limited to the foregoing effects, and other non-mentioned effects will be clearly understood by those skilled in the art from the description below.

### BRIEF DESCRIPTION OF THE DRAWINGS

Various aspects are described with reference to the drawings, and herein, like reference numerals are generally used to designate like constituent elements. In the exemplary embodiment below, for the purpose of description, a plurality of specific and detailed matters is suggested in order to provide general understanding of one or more aspects. However, it is apparent that the aspect(s) may be carried out without the specific and detailed matters. In other examples, well-known structures and devices are illustrated in a block diagram in order to facilitate describing one or more aspects.

FIG. 1 is a diagram illustrating an example of a system for performing a method of calculating a freshness score of a food ingredient according to exemplary embodiments of the present disclosure.

FIG. 2 is a flowchart for describing an example of a method of generating, by a server, monitoring information according to exemplary embodiments of the present disclosure.

FIG. 3 is a flowchart for describing an example of a method of calculating, by the server, a freshness score according to exemplary embodiments of the present disclosure.

FIG. 4 is a flowchart for describing an example of a method of re-calculating, by the server, a freshness score according to exemplary embodiments of the present disclosure.

FIG. 5 is a diagram illustrating an example of a system for performing a method of calculating a freshness score of a food ingredient according to exemplary embodiments of the present disclosure.

FIG. 6 is a general schematic diagram illustrating an example of a computing environment in which exemplary embodiments of the present disclosure are implementable.

### DETAILED DESCRIPTION

Various exemplary embodiments and/or aspects will be now disclosed with reference to drawings. In the following description, for the purpose of a description, multiple detailed matters will be disclosed in order to help comprehensive appreciation of one or more aspects. However, those skilled in the art of the present disclosure will recognize that the aspect(s) can be executed without the detailed matters. In the following disclosure and the accompanying drawings, specific exemplary aspects of one or more aspects will be described in detail. However, the aspects are exemplary and some of various methods in principles of various aspects may be used and the descriptions are intended to include all of the aspects and equivalents thereof. Specifically, in “embodiment”, “example”, “aspect”, “illustration”, and the like used in the specification, it may not be construed that a predetermined aspect or design which is described is more excellent or advantageous than other aspects or designs.

Hereinafter, like reference numerals refer to like or similar elements regardless of reference numerals and a duplicated description thereof will be omitted. Further, in describing an exemplary embodiment disclosed in the present disclosure, a detailed description of related known technologies will be omitted if it is determined that the detailed description makes the gist of the exemplary embodiment of the present disclosure unclear. Further, the accompanying drawings are only for easily understanding the exemplary embodiment disclosed in this specification and the technical spirit disclosed by this specification is not limited by the accompanying drawings.

Although the terms “first”, “second”, and the like are used for describing various elements or components, these elements or components are not confined by these terms, of course. These terms are merely used for distinguishing one element or component from another element or component. Therefore, a first element or component to be mentioned below may be a second element or component in a technical spirit of the present disclosure.

Unless otherwise defined, all terms (including technical and scientific terms) used in the present specification may be used as the meaning which may be commonly understood by the person with ordinary skill in the art, to which the present invention pertains. Terms defined in commonly used dictionaries should not be interpreted in an idealized or excessive sense unless expressly and specifically defined.

Moreover, the term “or” is intended to mean not exclusive “or” but inclusive “or”. That is, when not separately specified or not clear in terms of a context, a sentence “X uses A or B” is intended to mean one of the natural inclusive substitutions. That is, the sentence “X uses A or B” may be applied to any of the case where X uses A, the case where X uses B, or the case where X uses both A and B. Further, it should be understood that the term “and/or” used in this specification designates and includes all available combinations of one or more items among enumerated related items.

In addition, the word “comprises” and/or “comprising” means that the corresponding feature and/or component is present, but it should be appreciated that presence or addition of one or more other features, components, and/or a group thereof is not excluded. Further, when not separately specified or it is not clear in terms of the context that a singular form is indicated, it should be construed that the singular form generally means “one or more” in this specification and the claims.

Further, the terms “information” and “data” used in the specification may also be often used to be exchanged with each other.

It should be understood that, when it is described that a component is “connected to” or “accesses” another component, the component may be directly connected to or access the other component or a third component may be present therebetween. In contrast, it should be understood that, when it is described that a component is “directly connected to” or “directly access” another component, no component is present between the component and another component.

Suffixes “module” and “unit” for components used in the following description are given or mixed in consideration of easy preparation of the specification only and do not have their own distinguished meanings or roles.

The objects and effects of the present disclosure, and technical constitutions of accomplishing these will become obvious with reference to exemplary embodiments to be described below in detail along with the accompanying drawings. In describing the present disclosure, a detailed description of known function or constitutions will be omitted if it is determined that it unnecessarily makes the gist of the present disclosure unclear. In addition, terms to be described below as terms which are defined in consideration of functions in the present disclosure may vary depending on the intention or a usual practice of a user or an operator.

However, the present disclosure is not limited to exemplary embodiments disclosed below but may be implemented in various different forms. However, the exemplary embodiments are provided to make the present disclosure be complete and completely announce the scope of the present disclosure to those skilled in the art to which the present disclosure belongs and the present disclosure is just defined by the scope of the claims. Accordingly, the terms need to be defined based on contents throughout this specification.

In the present disclosure, the server may receive food ingredient information about food ingredients loaded into a moving object and environment information about an environment inside the moving object from a user terminal. The moving object may be a vehicle, a motorcycle, a ship, an airplane, and the like. The user terminal may be a terminal which is provided inside the moving object, and includes a sensing unit for sensing an internal environment, a communication unit for transmitting the sensed environment information to the server, and the like. The user terminal may include an Internet of Things (IoT) sensor which is capable of sensing the internal environment of the moving object and transmitting the sensed internal environment to the server. Further, the user terminal may include a display unit on which monitoring information, which is to be described below, is displayed, and a user input unit for transmitting a feedback on the monitoring information. That is, the user terminal may be a terminal which is loaded into a loading box of the moving object, senses the internal environment of the moving object, and also transmits monitoring information to the user.

According to the exemplary embodiment, the user terminal may include a first user terminal loaded into the loading box of the moving object, and a second user terminal possessed by a user. The first user terminal may include an IoT sensor which is capable of sensing the internal environment of the moving object and transmitting the sensed internal environment to the server. Otherwise, the first user terminal may also transmit the sensed environment information to the second user terminal. The second user terminal may include a display unit on which the monitoring information is displayed, and a user input unit for transmitting a feedback on the monitoring information.

The food ingredient information may be information about food ingredients loaded into the moving object. For example, the food ingredient information may be information indicating what kind of food ingredients are loaded into the moving object, such as fish, fruit, sweets, or retort products. As another example, the food ingredient information may be information indicating whether the food ingredient loaded into the moving object is a refrigerated product, a frozen product, or a warm product.

The environment information may include temperature data obtained by measuring a temperature inside the moving object, or humidity data obtained by measuring humidity inside the moving object. The server may receive environment information at a predetermined time interval.

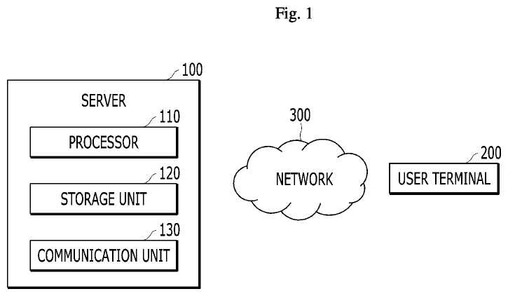

When the food ingredient information and the environment information are received, the server may calculate a freshness score of the food ingredient based on the food ingredient information and the environment information. The freshness score may be quantitative information indicating how fresh the food ingredient loaded inside the moving object is maintained or stored. It can be understood that as the freshness score is higher, the food ingredients loaded into the moving object is maintained in a fresher state. The server may generate monitoring information to be provided to the user terminal by using the calculated freshness score. The monitoring information may be information for visualizing the freshness score and transmitting the visualized freshness score to the user. When the user receives the monitoring information through the user terminal, the user may input a feedback on the freshness score. In this case, the server may update a weight for calculating the freshness score based on the feedback information received from the user terminal. Hereinafter, a method of calculating a freshness score of a food ingredient according to the present disclosure will be described with reference to FIGS. 1 to 5.

FIG. 1 is a diagram illustrating an example of a system for performing a method of calculating a freshness score of a food ingredient according to exemplary embodiments of the present disclosure.

Referring to FIG. 1, a server 100 may include a processor 110, a storage unit 120, and a communication unit 130. However, since the above-described constituent elements are not essential in implementing the server 100, the server 100 may have more or fewer components than those listed above.

The server 100 may include a predetermined type of computer system or computer device, for example, a microprocessor, a mainframe computer, a digital processor, a computing device, a portable device, and a device controller.

The processor 110 may control the overlap operation of the server 100. The processor 110 may provide appropriate information or function or process appropriate information or function by processing signals, data, information, and the like input or output through the constituent elements of the server 100 or driving an application program stored in the memory.

The processor 110 may be formed of one or more cores, and may include a processor, such as a central processing unit (CPU), a general purpose graphics processing unit (GPGPU), and a tensor processing unit (TPU) of the server 100, for performing a data analysis.

The processor 110 may calculate a freshness score for a food ingredient based on the food ingredient information and the environment information received through the communication unit 130.

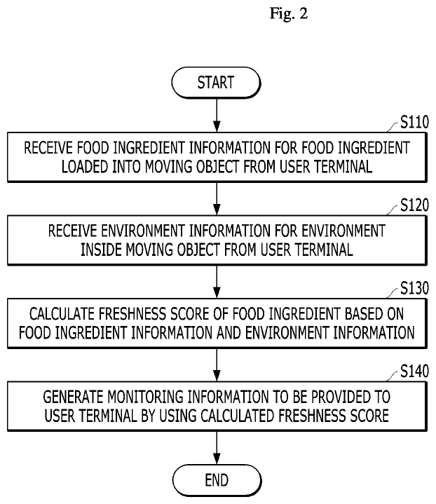

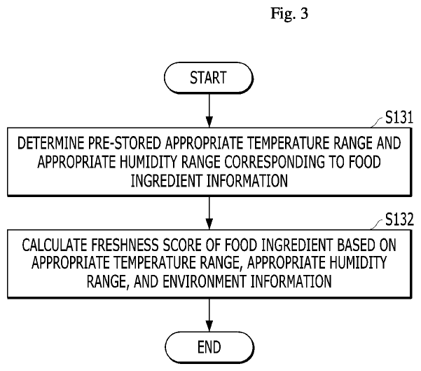

In particular, the processor 110 may determine a pre-stored appropriate temperature range and appropriate humidity range corresponding to the food ingredient based on the food ingredient information. The appropriate temperature range and the appropriate humidity range corresponding to the food ingredient may be pre-stored in the storage unit 120. When the appropriate temperature range is determined, the processor 110 may calculate a freshness score based on temperature data included in the appropriate temperature range and the environment information. For example, the communication unit 130 may receive the environment information at a predetermined time interval. The environment information may include temperature data obtained by measuring a temperature inside the moving object. The processor 110 may calculate the freshness score in such a way that the ratio in which at least one temperature data is included in the appropriate temperature range is higher, a higher score is determined. The environment information received at a predetermined time interval through the communication unit 130 may include humidity data obtained by measuring humidity inside the moving object. When the appropriate humidity range is determined, the processor 110 may calculate a freshness score based on the appropriate humidity range and at least one humidity data. The processor 110 may calculate the freshness score in such a way that the ratio in which at least one humidity data included in the appropriate humidity range is higher, a higher score is determined Hereinafter, the method of calculating, by the processor 110, the freshness score will be described with reference to FIGS. 2 and 3.

The storage unit 120 may include a memory and/or a persistent storage medium. The memory may include at least one type of storage medium among a flash memory type, a hard disk type, a multimedia card micro type, a card type of memory (for example, an SD or XD memory), a Random Access Memory (RAM), a Static Random Access Memory (SRAM), a Read-Only Memory (ROM), an Electrically Erasable Programmable Read-Only Memory (EEPROM), a Programmable Read-Only Memory (PROM), a magnetic memory, a magnetic disk, and an optical disk.

The storage unit 120 may include one or more memories including a buffer cache. Herein, the memory is a main storage device which a processor 110 directly accesses, such as a RAM including a DRAM and a SRAM, and may mean a volatile storage device in which stored information is instantly erased when a power supply is turned off, but is not limited thereto. The memory may be operated by the processor 110. The memory includes the buffer cache, and data may be stored in a data block of the buffer cache. The data may be recorded in the storage unit 120 by a background process.

The storage unit 120 may store a predetermined type of information generated or determined by the processor 110 and a predetermined type of information received by the communication unit 130. The storage unit 120 may store the food ingredient information and the environment information received through the communication unit 130. For example, the communication unit 130 may receive the environment information about the environment inside the moving object from the user terminal 200 at a predetermined time interval. The environment information may include temperature data obtained by measuring a temperature inside the moving object or humidity data obtained by measuring humidity inside the moving object, and accordingly, the storage unit 120 may store at least one temperature data and at least one humidity data. The storage unit 120 may store information on an appropriate temperature range and an appropriate humidity range corresponding to each of the plurality of food ingredients. The storage unit 120 may store a freshness score calculated by the processor 110 and feedback information received from the user terminal 200.

The communication unit 130 may include one or more modules capable of establishing communication between the server 100 and a communication system, between the server 100 and the user terminal 200, or the server 100 and a network. The communication unit 130 may include at least one of a wired Internet module and a wireless Internet module.

The communication unit 130 may receive food ingredient information about the food ingredient loaded into the moving object from the user terminal 200. The communication unit 130 may receive the environment information about the environment inside the moving object from the user terminal 200 at a predetermined time interval.

The user terminal 200 may include a Personal Computer (PC), a notebook computer, a mobile terminal, a smart phone, a tablet PC, a web-cam, and the like possessed by a user or provided in the moving object, and may include all kinds of terminals which are capable of accessing a wired/wireless network. However, the present disclosure is not limited thereto.

In the present disclosure, the user may input the food ingredient information about the food ingredient loaded into the moving object through the user terminal 200. The user terminal 200 may transmit the input food ingredient information to the server 100. The user terminal 200 may generate environment information by sensing the internal environment of the moving object and transmit the generated environment information to the server 100.

The user terminal 200 may receive monitoring information generated by using the freshness score of the food ingredient from the server 100. The user terminal 200 may provide the user with information on freshness of the food ingredient in real time by displaying the received monitoring information. Accordingly, the user may check the freshness of the food ingredient through the monitoring information that is changed in real time.

The network 300 in the present disclosure may be configured regardless of its communication mode, such as a wired mode and a wireless mode, and may be configured of various communication networks, such as a Personal Area Network (PAN), Local Area Network (LAN), and a Wide Area Network (WAN). Further, the network may be the publicly known World Wide Web (WWW), and may also use a wireless transmission technology used in PAN, such as Infrared Data Association (IrDA) or Bluetooth. The technologies described in the present specification may be used in other networks, as well as the foregoing networks.

According to the foregoing configuration, the server 100 may calculate a freshness score of the food ingredient based on the food ingredient information and the environment information received from the user terminal 200. The server 100 may generate the monitoring information to be provided to the user terminal 200 by using the calculated freshness score. According to the exemplary embodiment, the server 100 may receive the environment information at a predetermined time interval, and accordingly, the server 100 may generate the monitoring information at the predetermined time interval and transmit the generated monitoring information to the user terminal 200. Accordingly, the user may check the freshness of the food ingredient through the monitoring information that is changed in real time.

Hereinafter, a particular method of generating, by the server 100, monitoring information will be described.

FIG. 2 is a flowchart for describing an example of a method of generating, by the server, monitoring information according to exemplary embodiments of the present disclosure.

Referring to FIG. 2, the communication unit 130 of the server 100 may receive food ingredient information about the food ingredient loaded into a moving object from the user terminal 200 (S110).

The food ingredient information may be information for identifying the food ingredient loaded into the moving object. For example, the food ingredient information may be information indicating what kind of food ingredients are loaded into the moving object, such as fish, fruit, sweets, or retort products. As another example, the food ingredient information may be information indicating whether the food ingredient loaded into the moving object is a refrigerated product, a frozen product, or a warm product.

According to exemplary embodiments of the present disclosure, the processor 110 may determine a pre-stored appropriate temperature range and appropriate humidity range corresponding to the food ingredient based on the food ingredient information when the food ingredient information is received. The appropriate temperature range and appropriate humidity range may be the information pre-stored in the storage unit 120. The appropriate temperature range may be the range indicating a temperature appropriate to storing the food ingredient. For example, the storage unit 120 may store information indicating that the appropriate temperature range of fish is −10° C. to 5° C. The appropriate humidity range may be the range indicating humidity appropriate to store the food ingredient. For example, the storage unit 120 may store information indicating that the appropriate humidity range of fish is 0 to 50%.

The communication unit 130 may receive environment information about an environment inside the moving object from the user terminal 200 (S120). The environment information may include at least one of temperature data obtained by measuring a temperature inside the moving object and humidity data obtained by measuring humidity inside the moving object.

According to the exemplary embodiments of the present disclosure, the communication unit 130 may receive the environment information at a predetermined time interval. When the environment information is received at the predetermined time interval through the communication unit 130, the storage unit 120 may store at least one temperature data or at least one humidity data.

According to the exemplary embodiments of the present disclosure, the communication unit 130 may receive the environment information when a predetermined condition is satisfied. The predetermined condition may be, for example, the case where the environment information is generated by the user terminal 200. For example, the communication unit 130 may receive a signal indicating that sensing of the environment inside the moving object is completed from the user terminal 200. In this case, the communication unit 130 may receive the environment information from the user terminal 200. The predetermined condition may be, for example, the case where the processor 110 completes the calculation of the freshness score based on first environment information. When the calculation of the freshness score based on the first environment information is completed by the processor 110, the communication unit 130 may receive second environment information.

The processor 110 may calculate a freshness score of the food ingredient based on the food ingredient information and the environment information (S130).

The freshness score may be a score indicating how fresh the food ingredient loaded inside the moving object is maintained or stored. It can be understood that as the freshness score is higher, the food ingredients loaded into the moving object is maintained in a fresher state.

According to the exemplary embodiments of the present disclosure, the processor 110 may determine a pre-stored appropriate temperature range and appropriate humidity range corresponding to the food ingredient based on the food ingredient information after the environment information is received. The processor 110 may calculate a freshness score of the food ingredient based on the determined appropriate temperature range and appropriate humidity range, and the environment information.

For example, the processor 110 may calculate the freshness score by using a rule-based freshness model which calculates a first score based on a ratio in which at least one temperature data is included in the appropriate temperature range and a ratio in which at least one humidity data is included in the appropriate humidity range. The rule-based freshness model may be a neural network-based model pre-trained so as to calculate the first score based on the ratio in which at least one temperature data is included in the appropriate temperature range and the ratio in which at least one humidity data is included in the appropriate humidity range. The rule-based freshness model may be an algorithm that calculates the first score based on the ratio in which at least one temperature data is included in the appropriate temperature range and the ratio in which at least one humidity data is included in the appropriate humidity range. When the first score is calculated through the rule-based freshness model, the processor 110 may calculate the freshness score through a computation of the first score and a first weight preset for the first score. For example, the processor 110 may determine a value obtained by multiplying the first score and the first weight as the freshness score. Hereinafter, a method of calculating the first score by using the rule-based freshness model by the processor 110 will be described in more detail with reference to FIG. 3.

As another example, the processor 110 may calculate the freshness score by using a clustering-based freshness model which calculates a second score based on a density in which at least one temperature data is included in the appropriate temperature range and a density in which at least one humidity data is included in the appropriate humidity range. The clustering-based freshness model may be a neural network-based model pre-trained so as to calculate the second score based on the density in which at least one temperature data is included in the appropriate temperature range and the density in which at least one humidity data is included in the appropriate humidity range. The clustering-based freshness model may be an algorithm that calculates the second score based on the density in which at least one temperature data is included in the appropriate temperature range and the density in which at least one humidity data is included in the appropriate humidity range. When the second score is calculated through the clustering-based freshness model, the processor 110 may calculate the freshness score through a computation of the second score and a second weight preset for the second score. For example, the processor 110 may determine a value obtained by multiplying the second score and the second weight as the freshness score. Hereinafter, a method of calculating the second score by using the clustering-based freshness model by the processor 110 will be described in more detail with reference to FIG. 3.

According to the exemplary embodiments of the present disclosure, the processor 110 may calculate the freshness score by using an ensemble model. The ensemble model may be a deep leaning model implemented by a combination of the rule-based freshness model and the clustering-based freshness model. The rule-based freshness model may calculate the first score by a method of deriving a higher score as the food ingredient is stored in the appropriate temperature range and the appropriate humidity range. The clustering-based freshness model may calculate the second score by a method of deriving a higher score as there are fewer abnormal data out of the normal category. When there is a lot of data in which the temperature or humidity is rises or falls rapidly, the clustering-based freshness model determines that the corresponding data is abnormal, so that the clustering-based freshness model may determine the second score to be low. The ensemble model may calculate the freshness score through the computation of the first score, the first weight, the second score, and the second weight. The processor 110 may generate monitoring information by using the calculated freshness score, and transmit the generated monitoring information to the user terminal 200 through the communication unit 130. Accordingly, the user is capable of maintaining the temperature and humidity range appropriate to store the food ingredient through the monitoring information transmitted to the user terminal 200 and also is capable of monitoring the temperature and the humidity so that the temperature and the humidity are not sharply changed. Hereinafter, an example of the monitoring information generated by the processor 110 will be described with reference to operation S140.

According to the exemplary embodiments of the present disclosure, the processor 110 may determine the pre-stored appropriate temperature range and appropriate humidity range corresponding to the food ingredient after the food ingredient information is received. That is, the processor 110 may determine the pre-stored appropriate temperature range and appropriate humidity range corresponding to the food ingredient after operation S110. In this case, the processor 110 may calculate the freshness score of the food ingredient based on the predetermined appropriate temperature range and appropriate humidity range and the environment information.

According to the exemplary embodiments of the present disclosure, the appropriate temperature range or the appropriate humidity range corresponding to the food ingredient may not be stored in the storage unit 120. When the processor 110 determines that the appropriate temperature range or the appropriate humidity range corresponding to the food ingredient are not stored, the processor 110 may access a database provided by the Ministry of Food and Drug Safety through the communication unit 130. The processor 110 may also determine the appropriate temperature range or the appropriate humidity range corresponding to the food ingredient through the database provided by the Ministry of Food and Drug Safety.

The processor 110 may generate monitoring information to be provided to the user terminal 200 by using the calculated freshness score (S140).

The monitoring information may be information for visualizing the freshness score and transmitting the visualized freshness score to the user. The monitoring information may be information for providing a freshness score to the user in a text format. The monitoring information may be information in a graph format in which the freshness score that changes depending on the environment inside the moving object is reflected in real time.

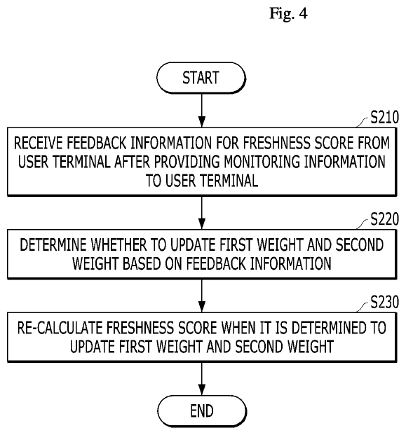

According to the exemplary embodiments of the present disclosure, the communication unit 130 may receive feedback information on the freshness score from the user terminal 200 after providing the monitoring information to the user terminal 200. The processor 110 may determine whether to update the first weight and the second weight based on the received feedback information. When the processor 110 determines to update the first weight and the second weight, the processor 110 may re-calculate the freshness score through the ensemble model. Hereinafter, the method of re-calculating the freshness score through the ensemble model by the processor 110 will be described with reference to FIG. 4.

According to the foregoing configuration, the processor 110 may generate monitoring information by using the freshness score calculated through the ensemble model, and transmit the generated monitoring information to the user terminal 200 through the communication unit 130. Accordingly, the user is capable of maintaining the temperature and humidity range appropriate to store the food ingredient through the monitoring information transmitted to the user terminal 200 and also is capable of monitoring the temperature and the humidity so that the temperature and the humidity are not sharply changed.

Hereinafter, an example of the calculation of the freshness score by the processor 110 will be described in more detail.

FIG. 3 is a flowchart for describing an example of a method of calculating, by the server, a freshness score according to exemplary embodiments of the present disclosure.

Referring to FIG. 3, the processor 110 of the server 100 may determine a pre-stored appropriate temperature range and appropriate humidity range corresponding to the food ingredient based on the food ingredient information (S131).

The appropriate temperature range and appropriate humidity range may be the information pre-stored in the storage unit 120. The appropriate temperature range may be the range indicating a temperature appropriate to storing the food ingredient. For example, the storage unit 120 may store information indicating that the appropriate temperature range of fish is −10° C. to 5° C. The processor 110 may determine the pre-stored appropriate temperature range corresponding to the food ingredient based on the food ingredient information. The appropriate humidity range may be the range indicating humidity appropriate to store the food ingredient. For example, the storage unit 120 may store information indicating that the appropriate humidity range of fish is 0 to 50%. The processor 110 may determine the pre-stored appropriate humidity range corresponding to the food ingredient based on the food ingredient information.

The processor 110 may calculate a freshness score of the food ingredient based on the appropriate temperature range, the appropriate humidity range, and the environment information (S132).

In particular, the processor 110 may calculate the freshness score by using the ensemble model including the rule-based freshness model that calculates the first score and the clustering-based freshness model that calculates the second score.

The rule-based freshness model may calculate the first score based on Equation 1 below. Equation 1 presents an algorithm for calculating an inclusion rate of the measured data, where a higher inclusion rate may indicate that the measured data is more likely to be included in the appropriate range.

$$i th Inclusion Rate ( \% ) = \frac{\# ( {T}_{lower} \leq {T}_{i} < {T}_{upper} and {H}_{lower} \leq {H}_{i} < {H}_{upper} )}{i} \qquad [ Equation 1 ]$$

Herein, i may be a natural number for indicating the time point or the number of times of the measurement. Inclusion rate may be a percentage value for indicating a ratio in which at least one temperature data is included in the appropriate temperature range and a ratio in which at least one humidity data is included in the appropriate humidity range. T_{lower} is a lower limit value of the appropriate temperature range, Tᵤₚₚₑᵣ is an upper limit value of the appropriate temperature range, and Tᵢ may be the temperature of the i^{th} measurement. H_{lower} is a lower limit value of the appropriate humidity range, Hᵤₚₚₑᵣ is an upper limit value of the appropriate humidity range, and Hᵢ may be the humidity of the i^{th} measurement.

The rule-based freshness model may determine a first inclusion rate by using first temperature data and first humidity data included in the first environment information among the environment information received at the predetermined time interval.

For example, when the first temperature data is included in the appropriate temperature range and the first humidity data is included in the appropriate humidity range, the first inclusion rate may be 100%. For another example, when the first temperature data is included in the appropriate temperature range and the first humidity data is not included in the appropriate humidity range, the first inclusion rate may be 50%. For another example, when the first temperature data is not included in the appropriate temperature range and the first humidity data is not included in the appropriate humidity range, the first inclusion rate may be 0%. The inclusion rate may be determined by repeating the measurement of the temperature data and the humidity data several times.

According to the exemplary embodiments of the present disclosure, the rule-based freshness model may determine the inclusion rate based on the first environment information and the second environment information received after the first environment information is received. For example, the rule-based freshness model may determine that the first inclusion rate is 100% and the second inclusion rate determined based on the second environment information is 50%. The rule-based freshness model may determine that the inclusion rate is 75% based on the first inclusion rate and the second inclusion rate.

The rule-based freshness model may determine a first section corresponding to the i^{th} inclusion rate among the plurality of sections that are divided into the predetermined number for calculating the first score. The rule-based freshness model may calculate the first score based on a rule pre-applied to the first section. Herein, the plurality of sections and information related to the rule applied to each of the plurality of sections may have been pre-stored in the storage unit 120.

For example, the first section among the plurality of sections may be a section corresponding to the case where the inclusion rate is 95% or more. The rule pre-applied to the first section may be the rule to determine the first score to 100 when the inclusion rate is 95% or more. The second section among the plurality of sections may be a section corresponding to the case where the inclusion rate is 50% or more and less than 95%. The rule pre-applied to the second section may be the rule to determine the first score in such a way that 0.5 points are deducted per 1% when the inclusion rate is 50% or more and less than 95%. The third section among the plurality of sections may be a section corresponding to the case where the inclusion rate is 0% or more and less than 50%. The rule pre-applied to the third section may be the rule to determine the first score in such a way that 0.5 points are deducted per 1% when the inclusion rate is 0% or more and less than 50%. When it is assumed that the first inclusion rate is determined to 70%, the rule-based freshness model may determine that the second section is the section corresponding to the first inclusion rate. Since the rule pre-applied to the second section is the rule to determine the first score in such a way that 0.5 points are deducted per 1% when the inclusion rate is 50% or more and less than 95%, the rule-based freshness model may determine the first score to 87.5 by performing the computation of 100-0.5×(95-70). The above-described examples are only sections and rules for helping understanding of the description, but the present disclosure is not limited thereto.

The clustering-based freshness model may determine a density-based cluster based on at least one temperature data and at least one humidity data, and calculate a second score based on a ratio of normal data present in the cluster among at least one temperature data and at least one humidity data. Herein, the cluster may be a cluster or a group of vector values expressed on a two-dimensional plane based on the temperature data and the humidity data. The clustering-based freshness model may be a model pre-trained to calculate the proportion of normal data in the total data as a percentage and determine the data outside the cluster deviating from the normal range as abnormal.

In particular, the normal data among at least one temperature data and at least one humidity data may be densely collected based on the cluster, and the abnormal data may be out of a normal range. The clustering-based freshness model may determine the second score based on the ratio of the abnormal data in the total data. The clustering-based freshness model may determine the second score in such a way that the more abnormal data there is, the lower the score is. For example, the clustering-based freshness model may express time series data as a two-dimensional vector with temperature and humidity as axes. The clustering-based freshness model may calculate an outlier score based on a relative density to each data point with a local outlier factor algorithm, and determine data exceeding a specific threshold as abnormal data. The data having a relatively larger density between the adjacent data may form the cluster and form a normal data cluster. The clustering-based freshness model may determine data having a relatively smaller density between the adjacent data as abnormal data. The clustering-based freshness model may calculate a ratio of normal data in the total data as a percentage and calculate a score. As the number of temperature and humidity data collected increases, the performance of the clustering-based freshness model using the local outlier factor method may be gradually improved. For example, as the number of temperature and humidity data collected increases, the local outlier factor, the threshold, and the like become accurate, so that the performance of the clustering-based freshness model using the local outlier factor method may be gradually improved.

When the first score and the second score are determined, the ensemble model may calculate a freshness score through the computation of the first score and the first weight preset for the first score, and the second score and the second weight preset for the second score. In particular, the ensemble model may calculate the freshness score based on Equation 2 below.

Freshness score=λ₁×first score+λ₂×second score [Equation 2]

Herein, λ₁ may be the first weight, and λ₂ may be the second weight. Depending on the exemplary embodiment, the value of the preset first weight may be 0.5, and the value of the preset second weight may be 0.5, and a sum of the first weight and the second weight may be 1. That is, in calculating the freshness score by the ensemble model, the first weight and the second weight may be preset so that the ratio of the first score and the ratio of the second score are the same. However, the first weight and the second weight may be updated based on the feedback information received from the user terminal 200. When the first weight and the second weight are updated, the ensemble model may re-calculate the freshness score. For example, the processor 110 may update the value of the first weight to 0.8, and update the value of the second weight to 0.2 based on the feedback information. The first score may be a score indicating whether the food ingredient is stored in the appropriate temperature range and the appropriate humidity range, and the second score may be a score indicating whether the temperature and the humidity inside the moving object are maintained without a sharp change. Accordingly, which factor is more focused on and considered to calculate the freshness score may be determined through the update of the weight. Hereinafter, the method of re-calculating the freshness score by the ensemble model when the processor 110 determines to update the first weight and the second weight will be described with reference to FIG. 4.

According to the foregoing configuration, the ensemble model may calculate the first score based on the rule-based freshness model and calculate the second score based on the clustering-based freshness model. The ensemble model may calculate the freshness score through the computation of the first score, the first weight, the second score, and the second weight. The rule-based freshness model may calculate the first score in such a way that the score increases as the food ingredient is stored in the appropriate temperature range and the appropriate humidity range. The clustering-based freshness model may calculate the second score in such a way that the score increases as there is less abnormal data out of the cluster. Therefore, when the processor 110 transmits the monitoring information generated by using the freshness score to the user terminal 200, the user is capable of maintaining the food ingredient in the appropriate temperature and humidity range and monitoring the temperature and the humidity so that the temperature and the humidity are not sharply changed.

Hereinafter, the method of re-calculating the freshness score by the ensemble model when the processor 110 determines to update the first weight and the second weight will be described.

FIG. 4 is a flowchart for describing an example of a method of re-calculating, by the server, a freshness score according to exemplary embodiments of the present disclosure.

Referring to FIG. 4, the communication unit 130 of the server 100 may receive feedback information on the freshness score from the user terminal 200 after providing the monitoring information to the user terminal 200 (S210).

The feedback information may be information about the evaluation of the freshness score by the user. For example, the feedback information may include information that the user rated as high, medium, or low for the freshness score. As another example, the feedback information may include a quantitative value for the freshness score input by the user.

The processor 110 may determine whether to update the first weight and the second weight based on the feedback information (S220).

In particular, the processor 110 may grant a penalty or a reward for the freshness score based on the feedback information in order to determine whether to update the first weight and the second weight.

For example, the communication unit 130 may receive first feedback information from a first user terminal. The first feedback information may include information evaluated by “high” for the freshness score. The processor 110 may grant a reward to the first weight and the second weight according to the evaluation of the user included in the first feedback information. The communication unit 130 may receive second feedback information from a second user terminal. The second feedback information may include information evaluated by “low” for the freshness score. The processor 110 may grant a penalty to the first weight and the second weight according to the evaluation of the user included in the second feedback information. When the ratio of the penalties is larger than the ratio of the rewards, the processor 110 may determine to update the first weight and the second weight. When the ratio of the rewards is larger than the ratio of the penalties, the processor 110 may determine not to update the first weight and the second weight. For another example, the communication unit 130 may receive the first feedback information from the first user terminal after providing first monitoring information. The first monitoring information may be information generated based on the first inclusion rate. The first feedback information may include information evaluated by “high” for the freshness score. The processor 110 may grant a reward to the first weight and the second weight according to the evaluation of the user included in the first feedback information. The communication unit 130 may transmit second monitoring information to the first user terminal after providing the first monitoring information. The second monitoring information may be information generated based on the second inclusion rate. The second inclusion rate may be information generated after the first inclusion rate is generated. The communication unit 130 may receive second feedback information from the first user terminal after providing the second monitoring information. The second feedback information may include information evaluated by “low” for the freshness score. The processor 110 may grant a penalty to the first weight and the second weight according to the evaluation of the user included in the second feedback information. When the ratio of the penalties is larger than the ratio of the rewards, the processor 110 may determine to update the first weight and the second weight. When the ratio of the rewards is larger than the ratio of the penalties, the processor 110 may determine not to update the first weight and the second weight.

When the processor 110 determines to update the first weight and the second weight, the processor 110 may re-calculate the freshness score through the ensemble model (S230).

In particular, the processor 110 may update the first weight and the second weight. Depending on the exemplary embodiment, the processor 110 may update the first weight and the second weight so that the sum of the updated first weight and the updated second weight is 1.

For example, the processor 110 may update the value of the first weight to 0.8 and update the value of the second weight to 0.2. For another example, the processor 110 may update the value of the first weight to 0.4 and update the value of the second weight to 0.6.

The processor 110 may re-calculate the freshness score through the ensemble model which performs the computation of the first score, the updated first weight, the second score, and the updated second weight. The processor 110 may generate monitoring information to be provided to the user terminal 200 by using the re-calculated freshness score.

According to the exemplary embodiments of the present disclosure, the re-calculated freshness score may include the first freshness score and the second freshness score.

In particular, the processor 110 may calculate the first freshness score through the ensemble model that performs the computation of the first score and a first-1 weight in which the first weight is updated, and the second score and a second-1 weight in which the second weight is updated. For example, the processor 110 may calculate the first freshness score by adding a value obtained by multiplying the first score and the first-1 weight and a value obtained by multiplying the second score and the second-1 weight. The processor 110 may calculate the second freshness score through the ensemble model that performs the computation of the first score and a first-2 weight in which the first weight is updated differently from the first-1 weight, and the second score and a second-2 weight in which the second weight is updated differently from the second-1 weight. For example, the processor 110 may calculate the first freshness score by adding a value obtained by multiplying the first score and the first-2 weight and a value obtained by multiplying the second score and the second-2 weight.

The first-2 weight may be a weight updated differently from a first ratio in which the first-1 weight is updated, and the second-2 weight may be a weight updated differently from a second ratio in which the second-1 weight is updated. For example, the processor 110 may update the value of the first-1 weight to 0.8, and update the value of the first-2 weight to 0.6. The processor 110 may update the value of the second-1 weight to 0.2, and update the value of the second-2 weight to 0.4.

In the exemplary embodiment of the present disclosure, the expressions, such as a first and a second, are used to distinguish the expressions modified by a first or a second, and the expressions, such as a first-1 and a first-2, or a second-1 and a second-2, are used to distinguish the expressions from each other.

The processor 110 may generate monitoring information to be provided to the user terminal 200 by using the first freshness score and the second freshness score when the first freshness score and the second freshness score are calculated. When the first freshness score and the second freshness score are calculated, the processor 110 may generate monitoring information in the form of AB test. Herein, the AB test may be a test for receiving a feedback from the user by using the freshness scores calculated in two versions. The processor 110 may receive feedback information indicating which freshness score between the first freshness score and the second freshness score matches the determination of the user by providing the monitoring information generated by using the first freshness score and the second freshness score. The processor 110 may re-calculate at least one freshness score between the first freshness score and the second freshness score based on the feedback information received through the communication unit 130.

For example, the communication unit 130 may receive first feedback information from a first user terminal. The first feedback information may include information evaluated by “high” for the first freshness score. The processor 110 may grant a reward to the first-1 weight and the second-1 weight according to the evaluation of the user included in the first feedback information. The communication unit 130 may receive second feedback information from a second user terminal. The second feedback information may include information evaluated by “low” for the freshness score. The processor 110 may grant a penalty to the first-2 weight and the second-2 weight according to the evaluation of the user included in the second feedback information. When the ratio of the penalties is larger than the ratio of the rewards, the processor 110 may determine to update at least one weight among the first-1 weight, the first-2 weight, the second-1 weight, and the second-2 weight. When the ratio of the rewards is larger than the ratio of the penalties, the processor 110 may determine not to update at least one weight among the first-1 weight, the first-2 weight, the second-1 weight, and the second-2 weight.

According to the foregoing configuration, the communication unit 130 may receive the feedback information for the freshness score from the user terminal 200 after providing the monitoring information to the user terminal 200. The processor 110 may re-calculate the freshness score based on the feedback information. For example, the processor 110 may re-calculate the freshness score by updating the first weight and the second weight. The processor 110 may re-generate monitoring information to be provided to the user terminal 200 by using the re-calculated freshness score. The communication unit 130 may receive feedback information for the re-calculated freshness score from the user terminal 200 by transmitting the re-generated monitoring information to the user terminal 200. As described above, since the processor 110 re-calculates the weight by reflecting the feedback of the user and calculate the freshness score through the re-calculated weight, it is possible to improve user satisfaction with the freshness score.

Hereinafter, an example of a system for performing a method of calculating, by the server, a freshness score of a food ingredient will be described.

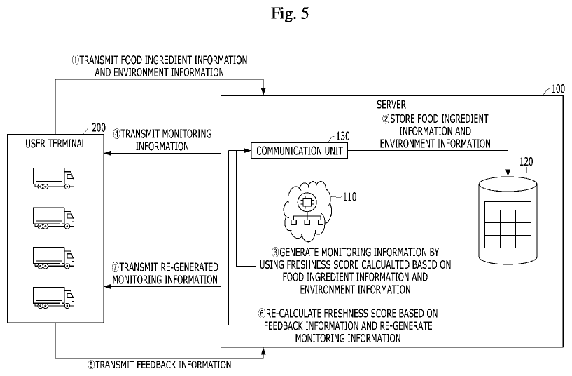

FIG. 5 is a diagram illustrating an example of a system for performing a method of calculating a freshness score of a food ingredient according to exemplary embodiments of the present disclosure.

Referring to FIG. 5, the user terminal 200 may transmit food ingredient information and environment information to the server 100. The storage unit 120 of the server 100 may store the food ingredient information and the environment information. The processor 110 may generate monitoring information by using a freshness score calculated based on the food ingredient information and the environment information. The communication unit 130 may transmit the generated monitoring information to the user terminal 200. The user terminal 200 may transmit feedback information generated based on an input of a user to the server 100. The processor 110 may re-calculate a freshness score based on the feedback information and re-generate monitoring information. The communication unit 130 may transmit the re-generated monitoring information to the user terminal 200.

Among the technical features expressed in FIG. 5, in order to avoid overlapping descriptions of the features described above, reference is made to the above-described contents, but a description of the content will be omitted in FIG. 5.

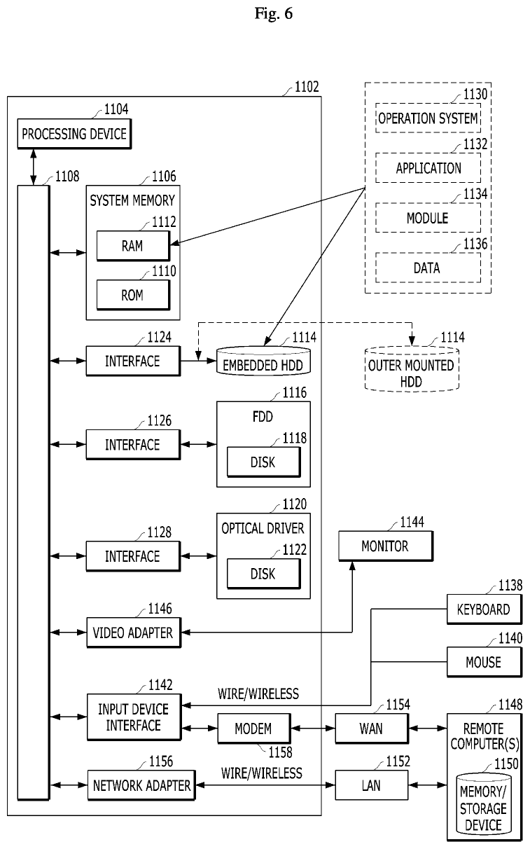

FIG. 6 is a general schematic view of an exemplary computing environment in which exemplary embodiments of the present disclosure may be implemented.

The present disclosure has generally been described above in association with a computer executable command which may be executed on one or more computers, but it will be well appreciated by those skilled in the art that the present disclosure can be implemented through a combination with other program modules and/or as a combination of hardware and software.

In general, the module in the present specification includes a routine, a procedure, a program, a component, a data structure, and the like that execute a specific task or implement a specific abstract data type. Further, it will be well appreciated by those skilled in the art that the method of the present disclosure can be implemented by other computer system configurations including a personal computer, a handheld computing device, microprocessor-based or programmable home appliances, and others (the respective devices may operate in connection with one or more associated devices as well as a single-processor or multi-processor computer system, a mini computer, and a main frame computer.

The exemplary embodiments described in the present disclosure may also be implemented in a distributed computing environment in which predetermined tasks are performed by remote processing devices connected through a communication network. In the distributed computing environment, the program module may be positioned in both local and remote memory storage devices.

The computer generally includes various computer readable media. The computer includes, as a computer accessible medium, volatile and non-volatile media, transitory and non-transitory media, and mobile and non-mobile media. As a non-limiting example, the computer readable media may include both computer readable storage media and computer readable transmission media.

The computer readable storage media include volatile and non-volatile media, transitory and non-transitory media, and mobile and non-mobile media implemented by a predetermined method or technology for storing information such as a computer readable instruction, a data structure, a program module, or other data. The computer readable storage media include a RAM, a ROM, an EEPROM, a flash memory or other memory technologies, a CD-ROM, a digital video disk (DVD) or other optical disk storage devices, a magnetic cassette, a magnetic tape, a magnetic disk storage device or other magnetic storage devices or predetermined other media which may be accessed by the computer or may be used to store desired information, but are not limited thereto.

The computer readable transmission media generally implement the computer readable instruction, the data structure, the program module, or other data in a carrier wave or a modulated data signal such as other transport mechanism and include all information transfer media. The term “modulated data signal” means a signal acquired by setting or changing at least one of characteristics of the signal so as to encode information in the signal. As a non-limiting example, the computer readable transmission media include wired media such as a wired network or a direct-wired connection and wireless media such as acoustic, RF, infrared and other wireless media. A combination of any media among the aforementioned media is also included in a range of the computer readable transmission media.

An exemplary environment 1100 that implements various aspects of the present disclosure including a computer 1102 is shown and the computer 1102 includes a processing device 1104, a system memory 1106, and a system bus 1108. The system bus 1108 connects system components including the system memory 1106 (not limited thereto) to the processing device 1104. The processing device 1104 may be a predetermined processor among various commercial processors. A dual processor and other multi-processor architectures may also be used as the processing device 1104.

The system bus 1108 may be any one of several types of bus structures which may be additionally interconnected to a local bus using any one of a memory bus, a peripheral device bus, and various commercial bus architectures. The system memory 1106 includes a read only memory (ROM) 1110 and a random access memory (RAM) 1112. A basic input/output system (BIOS) is stored in the non-volatile memories 1110 including the ROM, the EPROM, the EEPROM, and the like and the BIOS includes a basic routine that assists in transmitting information among components in the computer 1102 at a time such as in-starting. The RAM 1112 may also include a high-speed RAM including a static RAM for caching data, and the like.

The computer 1102 also includes an internal hard disk drive (HDD) 1114 (for example, EIDE and SATA)—the internal hard disk drive 1114 may also be configured for an external purpose in an appropriate chassis (not illustrated), a magnetic floppy disk drive (FDD) 1116 (for example, for reading from or writing in a mobile diskette 1118), and an optical disk drive 1120 (for example, for reading a CD-ROM disk 1122 or reading from or writing in other high-capacity optical media such as the DVD). The hard disk drive 1114, the magnetic disk drive 1116, and the optical disk drive 1120 may be connected to the system bus 1108 by a hard disk drive interface 1124, a magnetic disk drive interface 1126, and an optical disk drive interface 1128, respectively. An interface 1124 for implementing an external drive includes, for example, at least one of a universal serial bus (USB) and an IEEE 1394 interface technology or both of them.

The drives and the computer readable media associated therewith provide non-volatile storage of the data, the data structure, the computer executable instruction, and others. In the case of the computer 1102, the drives and the media correspond to storing of predetermined data in an appropriate digital format. In the description of the computer readable storage media, the mobile optical media such as the HDD, the mobile magnetic disk, and the CD or the DVD are mentioned, but it will be well appreciated by those skilled in the art that other types of storage media readable by the computer such as a zip drive, a magnetic cassette, a flash memory card, a cartridge, and others may also be used in an exemplary operating environment and further, the predetermined media may include computer executable instructions for executing the methods of the present disclosure.

Multiple program modules including an operating system 1130, one or more application programs 1132, other program module 1134, and program data 1136 may be stored in the drive and the RAM 1112. All or some of the operating system, the application, the module, and/or the data may also be cached in the RAM 1112. It will be well appreciated that the present disclosure may be implemented in operating systems which are commercially usable or a combination of the operating systems.

A user may input instructions and information in the computer 1102 through one or more wired/wireless input devices, for example, pointing devices such as a keyboard 1138 and a mouse 1140. Other input devices (not illustrated) may include a microphone, an IR remote controller, a joystick, a game pad, a stylus pen, a touch screen, and others. These and other input devices are often connected to the processing device 1104 through an input device interface 1142 connected to the system bus 1108, but may be connected by other interfaces including a parallel port, an IEEE 1394 serial port, a game port, a USB port, an IR interface, and others.

A monitor 1144 or other types of display devices are also connected to the system bus 1108 through interfaces such as a video adapter 1146, and the like. In addition to the monitor 1144, the computer generally includes other peripheral output devices (not illustrated) such as a speaker, a printer, others.

The computer 1102 may operate in a networked environment by using a logical connection to one or more remote computers including remote computer(s) 1148 through wired and/or wireless communication. The remote computer(s) 1148 may be a workstation, a server computer, a router, a personal computer, a portable computer, a micro-processor based entertainment apparatus, a peer device, or other general network nodes and generally includes multiple components or all of the components described with respect to the computer 1102, but only a memory storage device 1150 is illustrated for brief description. The illustrated logical connection includes a wired/wireless connection to a local area network (LAN) 1152 and/or a larger network, for example, a wide area network (WAN) 1154. The LAN and WAN networking environments are general environments in offices and companies and facilitate an enterprise-wide computer network such as Intranet, and all of them may be connected to a worldwide computer network, for example, the Internet.

When the computer 1102 is used in the LAN networking environment, the computer 1102 is connected to a local network 1152 through a wired and/or wireless communication network interface or an adapter 1156. The adapter 1156 may facilitate the wired or wireless communication to the LAN 1152 and the LAN 1152 also includes a wireless access point installed therein in order to communicate with the wireless adapter 1156. When the computer 1102 is used in the WAN networking environment, the computer 1102 may include a modem 1158, is connected to a communication server on the WAN 1154, or has other means that configure communication through the WAN 1154 such as the Internet, etc. The modem 1158 which may be an internal or external and wired or wireless device is connected to the system bus 1108 through the serial port interface 1142. In the networked environment, the program modules described with respect to the computer 1102 or some thereof may be stored in the remote memory/storage device 1150. It will be well known that an illustrated network connection is exemplary and other means configuring a communication link among computers may be used.

The computer 1102 performs an operation of communicating with predetermined wireless devices or entities which are disposed and operated by the wireless communication, for example, the printer, a scanner, a desktop and/or a portable computer, a portable data assistant (PDA), a communication satellite, predetermined equipment or place associated with a wireless detectable tag, and a telephone. This at least includes wireless fidelity (Wi-Fi) and Bluetooth wireless technology. Accordingly, communication may be a predefined structure like the network in the related art or just ad hoc communication between at least two devices.

The wireless fidelity (Wi-Fi) enables connection to the Internet, and the like without a wired cable. The Wi-Fi is a wireless technology such as the device, for example, a cellular phone which enables the computer to transmit and receive data indoors or outdoors, that is, anywhere in a communication range of a base station. The Wi-Fi network uses a wireless technology called IEEE 802.11 (a, b, g, and others) in order to provide safe, reliable, and high-speed wireless connection. The Wi-Fi may be used to connect the computers to each other or the Internet and the wired network (using IEEE 802.3 or Ethernet). The Wi-Fi network may operate, for example, at a data rate of 11 Mbps (802.11a) or 54 Mbps (802.11b) in unlicensed 2.4 and 5 GHz wireless bands or operate in a product including both bands (dual bands).

It may be appreciated by those skilled in the art that various exemplary logical blocks, modules, processors, means, circuits, and algorithm steps described in association with the exemplary embodiments disclosed herein may be implemented by electronic hardware, various types of programs or design codes (for easy description, herein, designated as “software”), or a combination of all of them. In order to clearly describe the intercompatibility of the hardware and the software, various exemplary components, blocks, modules, circuits, and steps have been generally described above in association with functions thereof. Whether the functions are implemented as the hardware or software depends on design restrictions given to a specific application and an entire system. Those skilled in the art of the present disclosure may implement functions described by various methods with respect to each specific application, but it should not be interpreted that the implementation determination departs from the scope of the present disclosure.

Various embodiments presented herein may be implemented as manufactured articles using a method, a device, or a standard programming and/or engineering technique. The term “manufactured article” includes computer programs or media which are accessible by a predetermined computer-readable device. For example, a computer readable storage media includes a magnetic storage device (for example, a hard disk, a floppy disk, a magnetic strip, or the like), an optical disk (for example, a CD, a DVD, or the like), a smart card, and a flash memory device (for example, an EEPROM, a card, a stick, a key drive, or the like), but is not limited thereto. The term “machine-readable media” includes a wireless channel and various other media that can store, possess, and/or transfer instruction(s) and/or data, but is not limited thereto.

The description of the presented embodiments is provided so that those skilled in the art of the present disclosure use or implement the present disclosure. Various modifications of the exemplary embodiments will be apparent to those skilled in the art and general principles defined herein can be applied to other exemplary embodiments without departing from the scope of the present disclosure. Therefore, the present disclosure is not limited to the exemplary embodiments presented herein, but should be interpreted within the widest range which is coherent with the principles and new features presented herein.

What is claimed is:

1. A method of calculating a freshness score of a food ingredient, the method being performed by a server including at least one processor, the method comprising:
receiving food ingredient information for food ingredients loaded into a moving object from a user terminal;
receiving environment information for an environment inside the moving object from the user terminal, the environment information including at least one of temperature data measured a temperature inside the moving object or humidity data measured humidity inside the moving object;
calculating a freshness score of the food ingredient based on the food ingredient information and the environment information; and
generating monitoring information to be provided to the user terminal by using the calculated freshness score.

2. The method of claim 1, wherein the environment information is received at a predetermined time interval or is received when a predetermined condition is satisfied.

3. The method of claim 1, wherein the calculating a freshness score of the food ingredient based on the food ingredient information and the environment information includes:
determining a pre-stored appropriate temperature range and appropriate humidity range corresponding to the food ingredient based on the food ingredient information; and
calculating the freshness score of the food ingredient based on the appropriate temperature range, the appropriate humidity range, and the environment information.

4. The method of claim 3, wherein the freshness score is determined by using a rule-based freshness model which calculates a first score based on a ratio in which at least one temperature data is included in the appropriate temperature range and a ratio in which at least one humidity data is included in the appropriate humidity range.

5. The method of claim 4, wherein the freshness score is calculated through a computation of the first score and a first weight preset for the first score.

6. The method of claim 4, wherein the rule-based freshness model calculates the first score based on Equation,
$$i th Inclusion Rate ( \% ) = \frac{\# ( {T}_{lower} \leq {T}_{i} < {T}_{upper} and {H}_{lower} \leq {H}_{i} < {H}_{upper} )}{i} ,$$
and
wherein the i is a natural number value for indicating measured time point or number of times, Inclusion rate is a percentage value for indicating a ratio in which the at least one temperature data is included in the appropriate temperature range and a ratio in which the at least one humidity data is included in the appropriate humidity range, T_{lower} is a lower limit value of the appropriate temperature range, Tᵤₚₚₑᵣ is an upper limit value of the appropriate temperature range, and Tᵢ is a temperature of i^{th} measurement, H_{lower} is a lower limit value of the appropriate humidity range, Hᵤₚₚₑᵣ is an upper limit value of the appropriate humidity range, and Hᵢ is humidity of i^{th} measurement.

7. The method of claim 6, wherein the rule-based freshness model determines a first section corresponding to the inclusion rate among plurality of sections divided into a predetermined number in order to calculate the first score, and
calculates the first score based on a rule pre-applied to the first section.

8. The method of claim 3, wherein the freshness score is determined based on a clustering-based freshness model which calculate a second score based on a density in which at least one temperature data is included in the appropriate temperature range and a density in which at least one humidity data is included in the appropriate humidity range.

9. The method of claim 8, wherein the freshness score is calculated through a computation of the second score and a second weight preset for the second score.

10. The method of claim 8, wherein the clustering-based freshness model determines a density-based cluster based on the at least one temperature data and the at least one humidity data, and calculates the second score based on a ratio of normal data present in the cluster among the at least one temperature data and the at least one humidity data included in the environment information.

11. The method of claim 3, wherein the freshness score is calculated by using an ensemble model, and
the ensemble model includes:
a rule-based freshness model which calculates a first score based on a ratio in which at least one temperature data is included in the appropriate temperature range and a ratio in which at least one humidity data is included in the appropriate humidity range; and
a clustering-based freshness model which calculates a second score based on a density in which the at least one temperature data is included in the appropriate temperature range and a density in which the at least one humidity data is included in the appropriate humidity range.

12. The method of claim 11, wherein the freshness score is calculated through a computation of the first score, a first weight preset for the first score, the second score, and a second weight preset for the second score.

13. The method of claim 12, further comprising:
receiving feedback information for the freshness score from the user terminal after providing the monitoring information to the user terminal;
determining whether to update the first weight and the second weight based on the feedback information; and
re-calculating the freshness score when it is determined to update the first weight and the second weight.

14. The method of claim 12, wherein the re-calculated freshness score includes:
a first freshness score calculated through a computation of the first score, a first-1 weight in which the first weight is updated, the second score, and a second-1 weight in which the second weight is updated; and
a second freshness score calculated through a computation of the first score, a first-2 weight in which the first weight is updated differently from the first-1 weight, the second score, and a second-2 weight in which the second weight is updated differently from the second-1 weight, and
the method further includes generating monitoring information to be provided to the user terminal by using the first freshness score and the second freshness score when the first freshness score and the second freshness score are calculated.

15. A method of calculating a freshness score of a food ingredient, the method being performed by a computing device including at least one processor, the method comprising:
receiving food ingredient information for food ingredients loaded into a moving object from a user terminal;
receiving environment information for an environment inside the moving object from the user terminal, the environment information including at least one of temperature data measured a temperature inside the moving object or humidity data measured humidity inside the moving object;
calculating a freshness score of the food ingredient based on the food ingredient information and the environment information; and
generating monitoring information to be provided to the user terminal by using the calculated freshness score.
